(19) United States
(12) Patent Application Publication
(10) Pub. No.: US 2026/0185990 A1
(43) Pub. Date: Jul. 2, 2026

(54) Use of substances for detecting exosomal CA125 in preparation of products for diagnosing or assisting in diagnosis of lung diseases

(71) Applicant: GUANGZHOU NATIONAL LABORATORY, Guangzhou, Guangdong (CN)

(72) Inventors: Ming DONG, Guangzhou, Guangdong (CN); Qian CHEN, Guangzhou, Guangdong (CN); Gaolei HU, Guangzhou, Guangdong (CN)

(21) Appl. No.: 18/852,030

(22) PCT Filed: Apr. 23, 2024
(86) PCT No.: PCT/CN2024/089354

§ 371 Date: Sep. 27, 2024

(30) Foreign Application Priority Data
May 15, 2023 (CN) 2023105475792

(51) Int. Cl. G01N 33/543; G01N 1/44; G01N 21/76; G01N 33/68

(52) U.S. Cl. CPC G01N 33/54333; G01N 1/44; G01N 21/763; G01N 33/6893; G01N 2333/47

(57) ABSTRACT

The present disclosure belongs to the technical field of biological detection and particularly relates to the use of a material for detecting exosomal CA125 in the preparation of a product for diagnosing or assisting in diagnosing a lung disease. The present disclosure provides an exosome treatment solution. By pretreating an exosome sample to be detected with the exosome treatment solution provided by the present disclosure, the influence of components in an eluent on the detection of the exosomal protein can be reduced, the detection time is effectively shortened, stable detection of the exosomal protein is realized, and fully automatic detection of the exosomal protein is facilitated.

### CROSS-REFERENCE TO RELATED APPLICATION

The present application is a national phase entry under 35 USC § 371 of International Application PCT/CN2024/089354, filed on Apr. 23, 2024, which claims the priority of Chinese Patent Application No. 2023105475792 filed on May 15, 2023. The priority applications are herein specifically incorporated by reference in their entirety for all purposes.

### TECHNICAL FIELD

The present disclosure belongs to the technical field of biological detection and particularly relates to the use of substances for detecting exosomal CA125 in the preparation of products for diagnosing or assisting in the diagnosis of lung diseases.

### BACKGROUND

Interstitial lung diseases (ILDs) are a group of heterogeneous diseases with progressive dyspnea and dry cough as the main symptoms, with lesions located in the lung parenchyma, and accompanied by extensive inflammation and diffuse fibrosis. Pulmonary fibrosis, as one of the common changes of interstitial lung diseases, is specifically characterized by alveolar epithelial cell injury, diffuse local alveolitis, pathological interstitial hyperplasia of fibroblasts, and deposition of extracellular matrix (ECM) in the pulmonary interstitial and alveoli, leading to irreversible structural remodeling and loss of function of lungs, impaired gas exchange and even respiratory failure. Early diagnosis of interstitial pneumonia and pulmonary fibrosis can guide medication use and improve prognosis. At present, commonly used clinical diagnostic methods include lung function tests, chest HRCT, invasive bronchoalveolar lavage fluid examination, and lung biopsy. Therefore, serological testing for early screening and development monitoring of ILD diseases has become a potential research direction in recent years.

Serum markers currently used clinically for assisting in diagnosis of ILDs in clinical practice include sialylated carbohydrate antigen (Krebs Von den Lungen-6, KL-6), but the detection rate and sensitivity of KL-6 are not high enough, so it has not been widely popularized. Carbohydrate antigen CA125 is currently the most widely used serum marker for tumors, which is mainly expressed in mesothelium, Müllerian duct epithelium, and epithelial tissues of body cavities with Müllerian duct derivatives formation. When pathological changes or abnormal hyperplasia occur in these tissues, the level of CA125 increases significantly. Many studies have confirmed that the CA125 positive rate can reach 60-70% in the serum of patients with lung cancer. Moreover, studies have confirmed that serum CA125 has a significant correlation with ILDs and can distinguish idiopathic pulmonary fibrosis (IPF) from other ILDs.

Exosomes refer to extracellular vesicles with a diameter of 30-150 nm and an average diameter of 100 nm, which are formed by fusion with other intracellular vesicles and organelles after invagination of plasma membrane. Exosomes carry abundant proteins, nucleic acids, lipids, and metabolites. By means of these materials, exosomes can become a medium of cell-to-cell communication by regulating downstream signal pathways in recipient cells.

Exosomes play a key role during the occurrence and development of diseases and have unique advantages and potential value in the diagnosis and treatment of diseases. Proteins in exosomes have also been extensively studied as potential biomarkers. CA125 is a glycoprotein expressed by MUC16 gene, which is present on cell membrane. Recent studies have found that the exosomal CA125 content in ovarian cancer is higher than the content of free CA125 in blood; however, the diagnostic application of exosomal CA125 protein in lung diseases has not been reported yet.

### SUMMARY

An object of a first aspect of the present disclosure is to provide an exosome treatment solution.

An object of a second aspect of the present disclosure is to provide uses of the exosome treatment solution of the first aspect of the present disclosure.

An object of a third aspect of the present disclosure is to provide a reagent.

An object of a fourth aspect of the present disclosure is to provide a kit.

An object of a fifth aspect of the present disclosure is to provide a detection system.

An object of a sixth aspect of the present disclosure is to provide a method for detecting the presence or content of an exosomal protein.

An object of a seventh aspect of the present disclosure is to provide uses of substances for detecting exosomal CA125 in preparation of products.

In order to achieve the above object, the technical solution used by the present disclosure is as follows:

According to the first aspect of the present disclosure, there is provided an exosome treatment solution comprising the following components: a buffer material, an ionic strength maintaining agent, a blocking agent, a surfactant, a chelating agent, and a sugar.

Preferably, the content of the buffer material in the exosome treatment solution is 2-4% by weight, further preferably 2.2-3.8%.

Preferably, the buffer material comprises at least one of borax salt, trimethylolaminomethane (Tris), a phosphate buffer salt, an acetate buffer salt, and a citrate buffer salt, further preferably trimethylolaminomethane (Tris) and a phosphate buffer salt.

Preferably, the content of the trimethylolaminomethane (Tris) in the exosome treatment solution is 0.9-2.1% by weight, further preferably 1-2%.

Preferably, the content of the phosphate buffer salt in the exosome treatment solution is 1.1-1.9% by weight, further preferably 1.2-1.8%.

Preferably, the phosphate buffer salt comprises at least one of sodium phosphate, potassium phosphate, dipotassium hydrogen phosphate, potassium dihydrogen phosphate, disodium hydrogen phosphate, and sodium dihydrogen phosphate, further preferably a mixture of disodium hydrogen phosphate and sodium dihydrogen phosphate.

Preferably, the weight ratio of disodium hydrogen phosphate to sodium dihydrogen phosphate is 1:(0.7-1.1), further preferably 1:(0.8-1).

Preferably, the disodium hydrogen phosphate is disodium hydrogen phosphate dodecahydrate.

Preferably, the sodium dihydrogen phosphate is sodium dihydrogen phosphate dihydrate.

Preferably, the ionic strength maintaining agent comprises a chloride, further preferably at least one of potassium chloride, sodium chloride, calcium chloride, and magnesium chloride, still further preferably sodium chloride.

Preferably, the content of the ionic strength maintaining agent in the exosome treatment solution is 0.6-1.2% by weight, further preferably 0.8-1%.

Preferably, the blocking agent comprises at least one of bovine serum albumin (BSA), casein, and collagen peptide.

Preferably, the blocking agent comprises at least one of casein and collagen peptide, and BSA.

Preferably, the blocking agent comprises BSA.

Preferably, the content of the blocking agent in the exosome treatment solution is 0.8-2.2% by weight, further preferably 1-2%.

Preferably, the surfactant comprises at least one of an anionic surfactant, a cationic surfactant, a zwitterionic surfactant, and a nonionic surfactant; further preferably a nonionic surfactant, e.g., the nonionic surfactant containing a polyoxyethylene alcohol structure (for example, an alcohol ethoxylate and a polyoxyethylene-polyoxyalkylene block copolymer), a fatty acid ester of polyoxyethylene sorbitol anhydride (for example, TWEEN (registered trademark) series (for example, Tween-20, Tween-40, and Tween-80)), polyoxyethylene octyl phenyl ether (for example, TRITON (registered trademark) series (for example, Triton X-100, Triton X-114, Triton X-305, Triton X-405, and Triton X-705)), and N-D-glucose-N-methylalkylamide (for example, MEGA series (for example, MEGA 8 and MEGA 10); still further preferably Tween-20.

Preferably, the content of the surfactant in the exosome treatment solution is 0.05-0.6% by weight, further preferably 0.1-0.5%.

Preferably, the chelating agent comprises at least one of ethylenediamine tetraacetic acid and a water-soluble salt of ethylenediamine tetraacetic acid, further preferably a water-soluble salt of ethylenediamine tetraacetic acid, still further preferably disodium ethylenediamine tetraacetate.

Preferably, the content of the chelating agent in the exosome treatment solution is 0.03-0.25% by weight, further preferably 0.05-0.2%.

Preferably, the sugar comprises at least one of sucrose and trehalose, further preferably trehalose.

Preferably, the content of the sugar in the exosome treatment solution is 0.8-2.2% by weight, further preferably 1-2%.

Preferably, the exosome treatment solution further comprises a preservative.

Preferably, the preservative comprises at least one of sodium azide, Proclin 300, 5-bromo-5-nitro-1,3-dioxane (BND), and Proclin 900, further preferably sodium azide or Proclin 300.

Preferably, the content of the preservative in the exosome treatment solution is 0.08-0.22% by weight, further preferably 0.1-0.2%.

Preferably, the exosome treatment solution further comprises water, and the amount of water is the balance.

Preferably, the pH of the exosome treatment solution is 6-8, further preferably 7-8, still further preferably 7.5.

Preferably, the exosome is derived from one of whole blood, serum, plasma, bronchoalveolar lavage fluid, and urine or a combination thereof.

According to the second aspect of the present disclosure, there are provided uses of the exosome treatment solution of the first aspect of the present disclosure in at least one of a1) to a6):
a1) detecting the presence of an exosomal material;
a2) detecting the content of an exosomal material;
a3) preparing a product for diagnosing or assisting in diagnosing a disease by means of the presence or content of an exosomal material;
a4) preparing a product for assessing the severity of a disease by means of the content of an exosomal material;
a5) preparing a product for detecting the presence of an exosomal material; and
a6) preparing a product for detecting the content of an exosomal material, wherein the material comprises at least one of a nucleic acid and a protein.

Preferably, the nucleic acid comprises at least one of a DNA and an RNA.

Preferably, the material comprises a protein.

Preferably, the product comprises at least one of a reagent, a kit, and a detection system.

Preferably, the use in a1) or a2) is for non-diagnostic purpose.

Preferably, the exosome is derived from one of whole blood, serum, plasma, bronchoalveolar lavage fluid, and urine or a combination thereof.

According to the third aspect of the present disclosure, there is provided a reagent comprising the exosome treatment solution of the first aspect of the present disclosure.

Preferably, the reagent is used for at least one of b1) to b4):
b1) detecting the presence of an exosomal material;
b2) detecting the content of an exosomal material;
b3) diagnosing or assisting in diagnosing a disease by means of the presence or content of an exosomal material; and
b4) assessing the severity of a disease by means of the content of an exosomal material.

The material comprises at least one of a nucleic acid and a protein.

Preferably, the nucleic acid comprises at least one of a DNA and an RNA.

Preferably, the material comprises a protein.

Preferably, the exosome is derived from one of whole blood, serum, plasma, bronchoalveolar lavage fluid, and urine or a combination thereof.

Preferably, the reagent is used for at least one of b1) to b4) by pretreating exosomes.

According to the fourth aspect of the present disclosure, there is provided a kit comprising at least one of cl) and c2):
c1) the exosome treatment solution of the first aspect of the present disclosure, and
c2) the reagent of the third aspect of the present disclosure.

Preferably, the kit is used for at least one of b1) to b4) of the third aspect of the present disclosure.

Preferably, the kit comprises at least one of cl) and c2), capture antibody-coated magnetic beads and a luminophore-labeled detection antibody:
c1) the exosome treatment solution of the first aspect of the present disclosure, and
c2) the reagent of the third aspect of the present disclosure;
the kit is used for at least one of d1) to d4):
d1) detecting the presence of an exosomal protein;
d2) detecting the content of an exosomal protein;
d3) diagnosing or assisting in diagnosing a disease by means of the presence or content of an exosomal protein; and
d4) assessing the severity of a disease by means of the content of an exosomal protein,
wherein the capture antibody and/or the detection antibody specifically bind to the protein.

Preferably, the capture antibody and the detection antibody are directed against different antigenic epitopes of the protein, respectively.

Preferably, the luminophore comprises at least one of acridinium ester, horseradish peroxidase, and alkaline phosphatase, further preferably acridinium ester.

Preferably, the kit further comprises at least one of a luminescent substrate and a protein calibrant, further preferably a luminescent substrate and a protein calibrant.

Preferably, the luminescent substrate comprises at least one of APS-5 or AMPPD buffer (matched with alkaline phosphatase), an acridinium ester pre-excitation solution and excitation solution (matched with acridinium ester), luminol, or isoluminol buffer (matched with horseradish peroxidase).

Preferably, the protein is CA125, and the kit is used for at least one of e1) to e5):
e1) detecting the presence of exosomal CA125;
e2) detecting the content of exosomal CA125;
e3) diagnosing or assisting in diagnosing a lung disease;
e4) assessing the severity of a lung disease; and
e5) diagnosing or assisting in diagnosing pulmonary fibrosis.

Preferably, the lung disease comprises pneumonia or an interstitial lung disease.

Preferably, the exosome is derived from one of whole blood, serum, plasma, bronchoalveolar lavage fluid, and urine or a combination thereof.

According to the fifth aspect of the present disclosure, there is provided a detection system comprising at least one of f1) to f3) and a chemiluminescence instrument:
f1) the exosome treatment solution of the first aspect of the present disclosure,
f2) the reagent of the third aspect of the present disclosure, and
f3) the kit of the fourth aspect of the present disclosure.

Preferably, the detection system is used for at least one of b1) to b4) in the third aspect of the present disclosure or at least one of d1) to d4) in the fourth aspect of the present disclosure.

According to the sixth aspect of the present disclosure, there is provided a method for detecting the presence or content of an exosomal protein, comprising a step of pretreating exosomes with the exosome treatment solution of the first aspect of the present disclosure.

Preferably, the step of pretreating the exosomes with the exosome treatment solution of the first aspect of the present disclosure specifically involves mixing the exosome treatment solution with the exosomes and reacting.

Preferably, the volume ratio of the exosome treatment solution to the exosomes is 1:2-30, for example, it may be 1:2, 1:5, 1:9, 1:10, 1:12, 1:15, 1:18, 1:20, 1:22, 1:25, 1:28, 1:30, etc.

Preferably, the reaction time is 5-120 min, further preferably 5-10 min, for example, it may be 5 min, 6 min, 7 min, 8 min, 9 min, 10 min, etc.

Preferably, the reaction temperature is 25-42° C., further preferably 35-40° C., for example, it may be 35° C., 36° C., 37° C., 38° C., 39° C., 40° C., etc.

Preferably, the exosome is derived from one of whole blood, serum, plasma, bronchoalveolar lavage fluid, and urine or a combination thereof.

Preferably, a preparation method for the exosomes is either by a fully automatic exosome extraction instrument or a manual extraction method (e.g., a centrifugation method, a kit method, an ultrafiltration method, a magnetic bead immunization method, and a polyethylene glycol precipitation method).

Preferably, the method further comprises the following steps:
(1) mixing the pretreated exosomes with capture antibody-coated magnetic beads, reacting to obtain a capture antibody-coated magnetic bead-exosome complex;
(2) mixing the capture antibody-coated magnetic bead-exosome complex with a luminophore-labeled detection antibody, reacting to obtain a capture antibody-coated magnetic bead-exosome-luminophore-labeled detection antibody complex; and
(3) mixing the capture antibody-coated magnetic bead-exosome-luminophore-labeled detection antibody complex with a luminescent substrate, reacting, and detecting the luminous intensity to obtain the protein content in an exosome sample.

Preferably, in steps (1) and (2), reaction time is 5-120 min, further preferably 5-10 min, for example, it may be 5 min, 6 min, 7 min, 8 min, 9 min, 10 min, etc.

Preferably, in steps (1) and (2), reaction temperature is 25-42° C., further preferably 35-40° C., for example, it may be 35° C., 36° C., 37° C., 38° C., 39° C., 40° C., etc.

Preferably, in step (3), a method for obtaining the protein content in the exosome sample involves using a protein calibrant to establish a standard curve by a standard curve method, and then calculating the protein content in the exosome sample according to the detected luminous intensity.

Preferably, the capture antibody-coated magnetic beads, the luminophore-labeled detection antibody, and the luminescent substrate are the capture antibody-coated magnetic beads, luminophore-labeled detection antibody, and luminescent substrate in the fourth aspect of the present disclosure.

Preferably, a preparation method for the capture antibody-coated magnetic beads is a common method in the art and specifically involves mixing activated magnetic beads with a capture antibody, and incubating and blocking the mixture.

Preferably, a mass ratio of the activated magnetic beads to the capture antibody is 100:1-10, further preferably 100:1-4, still further preferably 100:2-3.

Preferably, a preparation method for the activated magnetic beads involves mixing magnetic beads, EDC, and NHS, and reacting.

Preferably, a mass ratio of the magnetic beads to EDC to NHS is 0.03-0.07:0.06-0.14:1.

Preferably, the magnetic beads refer to a magnetic bead activation buffer containing magnetic beads.

Preferably, reaction condition is reaction at room temperature for 15-120 min.

Preferably, incubation time is 1˜4 h.

Preferably, the specific blocking method involves mixing an incubated mixed solution of the activated magnetic beads and the capture antibody with a blocking agent.

Preferably, amount of the incubated mixed solution of the activated magnetic beads and the capture antibody and the blocking agent by a mass ratio of the magnetic beads to the blocking agent is 1:2-10, further preferably 1:2-5.

Preferably, after blocking, the method further comprises the following step: removing the supernatant by magnetic separation, and then performing dilution with a diluent.

Preferably, the diluent consists of the following components in percentage by weight: 0.05-0.1% of Tris, 0.9-3.0% of NaCl, 1-3% of BSA, 0.1-0.5% of Tween-20, and 0.1-0.2% of sodium azide, with the balance being water, with pH 7.5.

Preferably, a preparation method for the luminophore-labeled detection antibody is a common method in the art and specifically involves mixing a luminophore with a detection antibody, and incubating and quenching the mixture.

Preferably, incubation time is 30-120 min.

Preferably, a specific method of quenching involves mixing an incubated mixed solution of the luminophore and a detection antibody with a quenching agent, and reacting.

Preferably, reaction time is 30-120 min.

Preferably, after quenching, the method further comprises the following steps: preforming a purification and diluting with a diluent.

Preferably, a method of purification includes preforming a dialysis or desalination column treatment.

Preferably, the diluent consists of the following components in percentage by weight: 0.05-0.1% of MES, 0.9-3.0% of NaCl, 1-3% of BSA, 0.1-0.5% of Tween-20, and 0.1-0.2% of sodium azide, with the balance being water, with pH 6.5.

Preferably, the method is for non-disease diagnostic purposes.

According to the seventh aspect of the present disclosure, there are provided uses of substances for detecting exosomal CA125 in the preparation of a product,
wherein the product is used for at least one of g1) to g3):
g1) diagnosing or assisting in diagnosing a lung disease;
g2) assessing the severity of a lung disease; and
g3) diagnosing or assisting in diagnosing pulmonary fibrosis.

Preferably, the lung disease comprises pneumonia or an interstitial lung disease.

Preferably, the substances for detecting CA125 comprises substances for detecting CA125 at the gene level and/or protein level, further preferably substances for detecting CA125 at the protein level.

Preferably, the product is a reagent, a kit, a detection chip, or a detection system.

Preferably, the product comprises the exosome treatment solution of the first aspect of the present disclosure.

Beneficial Effects of the present disclosure:

The present disclosure provides an exosome treatment solution. By pretreating the exosome sample to be detected with the exosome treatment solution provided by the present disclosure, the influence of components in the eluent on the detection of the exosomal protein can be reduced, the detection time is effectively shortened, stable detection of the exosomal protein is realized, which is conducive to the realization of fully automated detection of the exosomal protein.

The present disclosure discloses for the first time that exosomal CA125 is used as a marker for diagnosing or assisting in diagnosing lung diseases, assessing the severity of lung diseases, and/or diagnosing pulmonary fibrosis. Compared with the traditional marker (serum CA125), the method has a better diagnosis effect, i.e., a higher specificity and sensitivity, and has important clinical value and popularization prospect.

### BRIEF DESCRIPTION OF DRAWINGS

FIG. 1 is a graph showing the detection results of the content of exosomal CA125 protein by the detection methods provided by Comparative Example 1, Comparative Example 2, and Example 5 in Effect Example 2.

FIG. 2 is ROC curves of serum CA125 and exosomal CA125 for the diagnosis of the interstitial lung disease in Effect Example 3.

FIG. 3 is a graph showing the detection results of the content of exosomal CA125 protein in healthy human population, samples that are ILD positive and show negative result using commercially available serum KL-6 detection reagent, and samples that are ILD positive and show positive result using commercially available serum KL-6 detection reagent in Effect Example 4 by means of the detection method provided in Example 5.

### DETAILED DESCRIPTION

The content of the present disclosure will be further explained in detail by means of specific examples.

It should be understood that these examples are only used to illustrate the present disclosure but not to limit the scope of the present disclosure.

In the following examples, experimental methods for which no specific conditions are indicated are generally carried out under conventional conditions or conditions suggested by the manufacturer. Unless otherwise specified, the materials, reagents, etc. used in the examples are commercially available reagents and materials.

Abbreviations and key terms in these examples are defined as follows:
Tris: trimethylolaminomethane;
BSA: bovine serum albumin;
Tween-20: Tween 20;
EDTA-2Na: disodium ethylenediamine tetraacetate;
MES: morpholineethanesulfonic acid;
EDC: 1-ethyl-(3-dimethylaminopropyl) carbodiimide hydrochloride;
NHS: N-hydroxysuccinimide; and
BND: 5-bromo-5-nitro-1,3-dioxane.

### Example 1 Exosome Treatment Solution

The exosome treatment solution consisted of the following components in percentage by weight: 1% of Tris, 0.6% of disodium hydrogen phosphate dodecahydrate, 0.6% of sodium dihydrogen phosphate dihydrate, 0.8% of NaCl, 1% of BSA, 0.1% of Tween-20, 0.05% of EDTA-2Na, 1% of trehalose, and 0.1% of sodium azide, with the balance being water, with pH 7.5.

### Example 2 Exosome Treatment Solution

The exosome treatment solution consisted of the following components in percentage by weight: 2% of Tris, 1% of disodium hydrogen phosphate dodecahydrate, 0.8% of sodium dihydrogen phosphate dihydrate, 1% of NaCl, 2% of BSA, 0.5% of Tween-20, 0.2% of EDTA-2Na, 2% of trehalose, and 0.2% of sodium azide, with the balance being water, with pH 7.5.

### Example 3 Exosome Treatment Solution

The exosome treatment solution consisted of the following components in percentage by weight: 1.5% of Tris, 0.8% of disodium hydrogen phosphate dodecahydrate, 0.7% of sodium dihydrogen phosphate dihydrate, 0.9% of NaCl, 1.5% of BSA, 0.3% of Tween-20, 0.1% of EDTA-2Na, 1.5% of trehalose, and 0.15% of sodium azide, with the balance being water, with pH 7.5.

### Example 4 Exosome Treatment Solution

The exosome treatment solution consisted of the following components in percentage by weight: 1% of Tris, 0.6% of disodium hydrogen phosphate dodecahydrate, 0.6% of sodium dihydrogen phosphate dihydrate, 0.8% of NaCl, 1% of BSA, 0.1% of Tween-20, 0.05% of EDTA-2Na, 1% of trehalose, and 0.1% of Proclin 300, with the balance being water, with pH 7.5

### Example 5 Method for Detecting Exosomal CA125 Protein

The method for detecting exosomal CA125 protein comprised the following steps:

### (1) Preparation of Coated Magnetic Beads

1) Activation of magnetic beads: 10 mg of magnetic beads, an EDC solution (with a final concentration of 0.5 mg/mL) and an NHS solution (with a final concentration of 1 mg/mL) were added to 1 mL of a magnetic bead activation buffer (aqueous solution containing MES at a mass concentration of 1%) and reacted at room temperature for 30 min to activate the magnetic beads, and the supernatant was then removed by magnetic separation.
2) Antibody coupling: 10 mg of the magnetic beads were resuspended with 1 mL of magnetic bead coupling buffer (aqueous solution containing MES at a mass concentration of 1%), 300 μg of CA125 capture antibody (Beijing KeyGen, Cat. No.: BXA003) was added, and the mixture was incubated at room temperature for 2 h to obtain a solution of magnetic bead-antibody conjugate.
3) Blocking: 100 μL of blocking agent (aqueous solution of bovine serum albumin at a mass concentration of 1%) was added to the solution of the magnetic bead-antibody conjugate, and the mixture was incubated at room temperature for 2 h.
4) Cleaning: After the supernatant was removed by magnetic separation, 1 mL of magnetic bead cleaning solution (PBS buffer containing Proclin 300 at a mass concentration of 0.1%) was added for cleaning, and after the cleaning step was repeated twice, the supernatant was removed by magnetic separation to obtain CA125 capture antibody-coated magnetic beads (coated magnetic beads against CA125).
5) Preparation of coated magnetic bead working solution: The coated magnetic beads against CA125 were diluted with a diluent for coated magnetic beads to obtain a coated magnetic bead working solution with a concentration of 0.5 mg/mL, wherein the diluent for coated magnetic beads consisted of the following components in percentage by weight: 0.1% of Tris, 2% of NaCl, 3% of BSA, 0.5% of Tween-20, and 0.1% of sodium azide, with the balance being water, with pH 7.5.

### (2) Preparation of Acridinium Ester Marker

1) Preparation of acridinium ester mother liquor: Acridinium ester was dissolved in anhydrous DMSO to make its final concentration be 5 mg/mL.
2) Labeling reaction: 300 μg of CA125 detection antibody (Beijing KeyGen, Cat. No.: BXA004) was diluted with 0.2 M NaHCO₃ (pH=9.0) solution, and 15 μL of the acridinium ester mother liquor was added to make the volume constant at 300 μL. At this time, the concentration of acridinium ester was 0.25 mg/mL. It was well wrapped with tin foil, and the mixed solution was put on a constant temperature mixer for labeling at room temperature for 30 minutes.
3) Quenching reaction: 100 μL of labeling stop buffer (0.2 M NaHCO₃ solution containing lysine at a mass concentration of 10%, pH 9.0) was added and mixed uniformly at room temperature for 30 minutes.
4) Purification: The labeled antibody was purified by a desalting column, and the acridinium ester-labeled protein component was collected for concentration detection to obtain CA125 detection antibody with the acridinium ester marker.
5) Preparation of luminophore-labeled antibody working solution (acridinium ester marker working solution): The CA125 detection antibody with the acridinium ester marker was diluted with a diluent for acridinium ester markers to obtain an acridinium ester marker working solution with a concentration of 0.4 μg/mL, wherein the diluent for acridinium ester markers consisted of the following components in percentage by weight: 0.1% of MES, 2% of NaCl, 3% of BSA, 0.1% of Tween-20, and 0.1% of sodium azide, with the balance being water, with pH 6.5.

### (3) Pretreatment of Exosome Sample

1) Sample pretreatment: After serum or plasma was diluted 1× with PBS, it was filtered with a 0.22 μm filter membrane to remove cell debris and other impurities.
2) Exosome extraction: Exosomes were extracted by an automatic exosome extractor.

10 μL of the exosome sample was mixed with 90 μL of the exosome treatment solution of Example 1 and reacted at 37° C. for 5 min.

### (4) Detection and Calculation of CA125 Content in Exosome Sample to be Detected

1) 20 μL of the pretreated sample obtained in step (3) was taken, 50 μL of the coated magnetic bead working solution was added, and the mixture was mixed uniformly and incubated at 37° C. for 5 min; then, after magnetic separation, the unbound material was washed off, and the supernatant was discarded to obtain a coated magnetic bead-exosome complex.
2) 50 μL of the luminophore-labeled antibody working solution (acridinium ester marker working solution) was added to the coated magnetic bead-exosome complex, uniformly mixed, and incubated at 37° C. for 5 min; then, after magnetic separation, the unbound material was washed off, a coated magnetic bead-exosome-marker complex was obtained.
3) 100 μL of acridinium ester pre-excitation solution (aqueous solution containing 0.1 wt % of 30 wt % hydrogen peroxide and 0.2 mol/L nitric acid) and 100 μL of excitation solution (aqueous solution containing 0.1 wt % cetyltrimethylammonium chloride and 0.2 mol/L sodium hydroxide) were added to the obtained coated magnetic bead-exosome-marker complex, and after thorough mixing, the maximum luminous intensity was determined; and a standard curve was constructed based on the luminous intensities detected by standard samples, and the CA125 content in the exosome sample to be detected was calculated by the standard curve.

### Example 6 Method for Detecting Exosomal CA125 Protein

The detection method of this example was the same as that in Example 5, only except that the exosome treatment solution of Example 2 was used in step (3).

### Example 7 Method for Detecting Exosomal CA125 Protein

The detection method of this example was the same as that in Example 5, only except that the exosome treatment solution of Example 3 was used in step (3).

### Example 8 Method for Detecting Exosomal CA125 Protein

The detection method of this example was the same as that in Example 5, only except that the exosome treatment solution of Example 4 was used in step (3).

### Example 9 Method for Detecting Exosomal CA125 Protein

The detection method of this example was the same as that in Example 5, only except that the amount of the CA125 capture antibody in 2) of step (1) was 200 μg.

### Example 10 Method for Detecting Exosomal CA125 Protein

The detection method of this example was the same as that in Example 5, only except that the amount of the CA125 detection antibody in 2) of step (2) was 200 μg.

### Comparative Example 1 Method for Detecting Exosomal CA125 Protein

The detection method of this example was the same as that in Example 5, only except that the exosome sample was not pretreated, that is, step (3) was not comprised.

### Comparative Example 2 Method for Detecting Exosomal CA125 Protein

The detection method of this example was the same as that in Example 5, only except that the exosome treatment solution of Example 1 was replaced by a commercially available PBS buffer (Shanghai Beyotime Biotech Inc., Cat. No.: ST447).

### Effect Example 1

### Effect of Exosome Treatment Solution on Exosomal CA125 Protein Detection Result (Dilution Recovery)

A positive sample of serum exosomal CA125 was subjected to exosome extraction with a kit to obtain an exosome stock solution, which was diluted 2× and 4× with PBS buffer. Then, the detection methods of Examples 5-10 and Comparative Examples 1 and 2 were respectively used to detect the exosomal CA125 protein content on a fully automatic chemiluminescence immunoassay analyzer (model: shine i2910). The dilution recovery rate was calculated according to the luminescence values of the exosome stock solution, 2× dilution, and 4× dilution. After dilution, the sample was left at room temperature for more than 1 hour before being detected alongside the exosome stock solution.

The results were shown in Table 1. After pretreatment with the exosome treatment solution provided by the present disclosure (Examples 5-10), the exosomal protein was detected, the dilution recovery rate was 92-112%, which was significantly superior to that without pretreatment or that with pretreatment with PBS buffer (Comparative Examples 1 and 2). It could be seen that where the exosome sample to be detected was pretreated with the exosome treatment solution provided by the present disclosure, the exosome sample was diluted to reduce the interference of high salt level and the surfactant component in the elution solution on the exosomal protein detection, thereby facilitating the realization of the fully automatic detection of the exosomal protein.

TABLE 1
Effect of exosome treatment solution on exosomal CA125 protein detection results

| | Sample | Sample 2 × | | Sample 4 × | |
|---|---|---|---|---|---|
| | (Original) | diluted (PBS) | | diluted (PBS) | |
| | Average | Average | Dilution | Average | Dilution |
| | luminescence | luminescence | recovery | luminescence | recovery |
| | value | value | (2 × diluted/ | value | (4 × diluted/ |
| Group | (RLU) | (RLU) | original) | (RLU) | original) |
| Example 5 | 56743 | 29851 | 105% | 15320 | 108% |
| Example 6 | 54789 | 28875 | 105% | 15082 | 110% |
| Example 7 | 56274 | 27368 | 97% | 14784 | 105% |
| Example 8 | 49774 | 25087 | 101% | 13637 | 110% |
| Example 9 | 46783 | 24562 | 105% | 13045 | 112% |
| Example 10 | 43345 | 20645 | 95% | 9963 | 92% |
| Comparative | 51163 | 38043 | 149% | 23764 | 186% |
| Example 1 | | | | | |
| Comparative | 44176 | 33892 | 153% | 21352 | 193% |
| Example 2 | | | | | |

### Effect Example 2

### Effect of Exosome Pretreatment Solution on Significant Differences of Exosomal CA125 Protein Detection Results

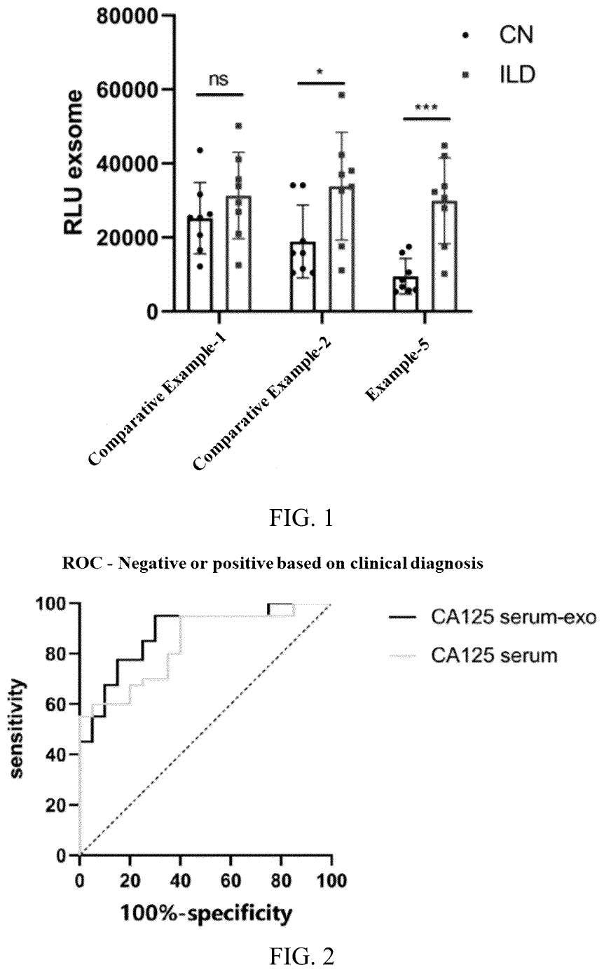

8 healthy human serum samples (CN) and 8 ILD-positive samples (ILD) were taken and detected for the content of exosomal CA125 protein by the detection methods provided in Comparative Example 1, Comparative Example 2, and Example 5, and the significant differences of the three methods was assessed. The results were shown in FIG. 1, in which no significant difference was found in the results obtained using the detection method of Comparative Example 1, the results obtained using the detection method of Comparative Example 2 shown significant difference (p<0.05), and the results obtained using the detection method of Example 5 shown extremely significant difference (p<0.001).

### Effect Example 3

### Comparison of Serum CA125 and Exosomal CA125 Detection Results Between Healthy Human Samples and ILD Patient Samples

20 serum samples from healthy human population, 20 samples that were ILD positive and showed negative result using a commercially available serum KL-6 detection reagent, and 20 samples that were ILD positive and showed positive result using the commercially available serum KL-6 detection reagent were taken. CA125 was detected in the 60 serum samples using a certified serum CA125 kit available on the market. The content of exosomal CA125 protein was detected by the detection method provided in Example 5. ROC analysis was carried out on the two parts of results, and the results were shown in FIG. 2. Whether the sample was negative or positive was defined based on the clinical diagnosis of interstitial lung disease. ROC curves were made separately, wherein the AUC of ROC curve for exosomal CA125 (serum-exo) was 0.88, and the AUC of ROC curve for serum CA125 (serum) was 0.83. It could be seen that exosomal CA125 (serum-exo) showed a superior diagnostic effect to serum CA125 (serum). According to AUC, the cutoff value of exosomal CA125 was 11000, with a sensitivity of 90%, and the cutoff value of serum CA125 was 115000, with a sensitivity of 70%. The sensitivity of exosomal CA125 measurement was higher than that of serum CA125 measurement, that is, using exosomal CA125 as a marker for lung diseases such as ILDs resulted in a higher sensitivity.

### Effect Example 4

### Comparison of Exosomal CA125 Detection Results Between Healthy Human Samples and ILD Patient Samples

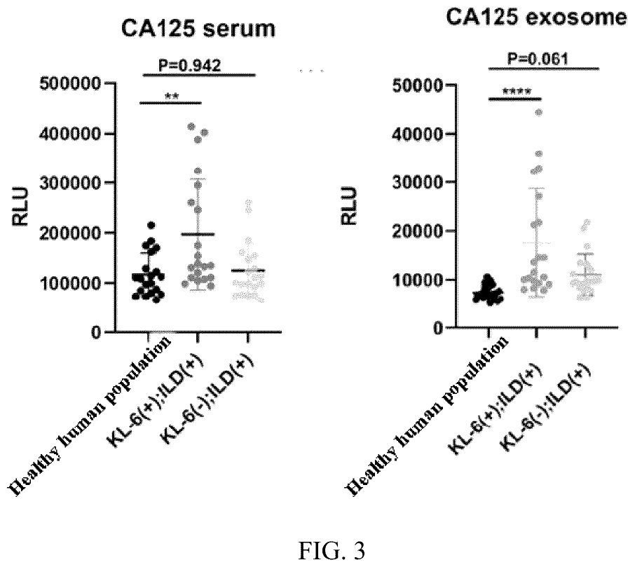

20 serum samples from healthy human population, 20 samples that were ILD positive and showed negative result using a commercially available serum KL-6 detection reagent, and 20 samples that were ILD positive and showed positive result using the commercially available serum KL-6 detection reagent were taken. The content of exosomal CA125 protein was detected by the detection method provided in Example 5. The results were shown in Table 2 and FIG. 3. For ILD patients with serum KL-6 positive, the exosomal CA125 protein detection luminescence values thereof were all significantly higher than the exosomal CA125 protein detection luminescence values of the samples from the healthy human population (P<0.0001), and the serum CA125 protein detection luminescence values thereof were all significantly higher than the exosomal CA125 protein detection luminescence values of the samples from the healthy human population (P<0.01) but significantly lower than the exosomal CA125 protein detection luminescence values. For ILD patients with serum KL-6 negative, the serum CA125 protein detection luminescence values thereof had no statistical difference from the serum CA125 protein detection luminescence values of the healthy human population (P=0.942), and the exosomal CA125 protein detection luminescence values thereof all tended to be higher than the exosomal CA125 protein detection luminescence values of the samples from the healthy human population (P=0.061). It could be seen that the exosomal CA125 detection could distinguish some of the samples from patients with ILD-positive and serum KL-6-negative from the samples from the healthy human population to some extent (the effect was superior to serum CA125) and had a better diagnostic effect.

TABLE 2
Comparison of exosomal CA125 detection results between
healthy human samples and ILD patient samples
Exosomal CA125 detection luminescence value

| Serum KL-6-positive | | Serum KL-6-negative | | Sample from healthy | |
|---|---|---|---|---|---|
| and ILD-positive | | and ILD-positive | | human population | |
| | Luminescence | | Luminescence | | Luminescence |
| No. | value | No. | value | No. | value |
| 25842 | 35975 | 7185 | 20511 | 1 | 7000 |
| 26628 | 11514 | 7367 | 6282 | 2 | 5180 |
| 7889 | 13644 | 7470 | 6270 | 3 | 10581 |
| 7892 | 9042 | 7467 | 16941 | 4 | 9030 |
| 27559 | 10573 | 7510 | 7772 | 5 | 7646 |
| 27542 | 27055 | 7637 | 12090 | 6 | 6040 |
| 7787 | 44444 | 7696 | 9331 | 7 | 8570 |
| 7384 | 8214 | 7726 | 9674 | 8 | 7070 |
| 27069 | 32900 | 7803 | 7700 | 9 | 6490 |
| 27681 | 14562 | 7856 | 11291 | 10 | 7110 |
| 7651 | 9783 | 7876 | 13092 | 11 | 8090 |
| 7770 | 14570 | 7875 | 9780 | 12 | 6920 |
| 7855 | 7950 | 7824 | 9261 | 13 | 9860 |
| 7885 | 10060 | 7818 | 9660 | 14 | 6220 |
| 22166 | 32290 | 7893 | 9814 | 15 | 6650 |
| 19119 | 10350 | 7862 | 8424 | 16 | 6290 |
| 27685 | 7830 | 7948 | 21826 | 17 | 5850 |
| 26756 | 9260 | 7952 | 8274 | 18 | 9466 |
| 7926 | 21280 | 7920 | 9952 | 19 | 7240 |
| 25225 | 21670 | 7928 | 13534 | 20 | 5480 |

### Effect Example 5

### Exosomal CA125 for Diagnosis of Interstitial Lung Disease

According to the exosomal CA125 luminescence values of the samples from the healthy human population and the ILD patients in Effect Example 3, it could be preliminarily determined that the cutoff value in the exosomal CA125 detection method was 11000. Whether the exosomal CA125 detection results of the samples from the ILD patients were negative or positive was determined based on the cutoff value, and the correlation between the negative or positive detection result and the clinical symptoms of the ILD patients was then assessed. The results were shown in Table 3. In conjunction with the test results in Tables 2 and 3, it was preliminarily determined that the cutoff value for exosomal CA125 was 11000, and for ILD patients whose results detected by the commercially available serum KL-6 detection kit were negative (<500 U/mL), the exosomal CA125 detection rate was 35% (7/20). This indicated that compared with the serum KL-6 detection kit, exosomal CA125 resulted in a higher detection rate of interstitial pneumonia in the serum KL-6 negative samples. In addition, by comparing the correlation between the measured values of exosomal CA125 and serum KL-6 and the clinical manifestations of the ILD-positive patients, among the 7 patients with serum KL-6-negative and CA125-positive, 1 patient showed respiratory failure and 1 patient had idiopathic interstitial pneumonia. Among the 11 patients whose exosomal CA125 and serum KL-6 were both positive, 8 patients were clinically diagnosed with pulmonary fibrosis and 3 patients showed severe pneumonia/respiratory failure, indicating that exosomal CA125 could be used as a marker for screening out patients with interstitial pneumonia who had developed pulmonary fibrosis or progressed to severe pneumonia. This showed that exosomal CA125 had important potential clinical value during the diagnosis of interstitial lung diseases and the assessment of the severity of interstitial lung diseases.

TABLE 3
Results of exosomal CA125 for diagnosis of interstitial lung diseases

| | Commercially | | | |
|---|---|---|---|---|
| | available | Exosomal | | Was pulmonary fibrosis, |
| | serum KL-6 | CA125 | | severe pneumonia or |
| | measurement | Luminescence | | respiratory failure |
| No. | value (U/mL) | value | Classification | present? |
| Preliminary | 500 | 11000 | | |
| cutoff | | | | |
| 7185 | 472 | 20511 | Serum KL-6 | Yes |
| 7467 | 277 | 16941 | negative and | Yes |
| 7637 | 174 | 12090 | exosomal | No |
| 7856 | 170 | 11291 | CA125 | No |
| 7876 | 229 | 13092 | positive | No |
| 7948 | 458 | 21826 | | Yes |
| 7928 | 149 | 13534 | | No |
| 25842 | 9674 | 35975 | Serum KL-6 | Yes |
| 26628 | 796 | 11514 | positive and | Yes |
| 7889 | 1115 | 13644 | exosomal | Yes |
| 27542 | 2991 | 27055 | CA125 | Yes |
| 7787 | 975 | 44444 | positive | Yes |
| 27069 | 10000 | 32900 | | Yes |
| 27681 | 3821 | 14562 | | Yes |
| 7770 | 2324 | 14570 | | No |
| 22166 | 5848 | 32290 | | No |
| 7926 | 3403 | 21280 | | Yes |
| 25225 | 7353 | 21670 | | Yes |

The above examples are preferred embodiments of the present disclosure, and the embodiments of the present disclosure are not limited by the above examples. Any other changes, modifications, substitutions, combinations and simplifications made without departing from the spirit and principle of the present disclosure shall be equivalent substitutions and are all included in the scope of protection of the present disclosure.

1. An exosome treatment solution comprising the following components: a buffer material, an ionic strength maintaining agent, a blocking agent, a surfactant, a chelating agent, and a sugar.

2. The exosome treatment solution according to claim 1, wherein the buffer material comprises at least one of borax salt, trimethylolaminomethane, a phosphate buffer salt, an acetate buffer salt, and a citrate buffer salt;
preferably, the ionic strength maintaining agent comprises a chloride salt;
preferably, the blocking agent comprises at least one of bovine serum albumin, casein, and collagen peptide;
preferably, the surfactant comprises at least one of an anionic surfactant, a cationic surfactant, a zwitterionic surfactant, and a nonionic surfactant;
preferably, the chelating agent comprises at least one of ethylenediamine tetraacetic acid and a water-soluble salt of ethylenediamine tetraacetic acid;
preferably, the sugar comprises at least one of sucrose and trehalose; and
preferably, the exosome treatment solution further comprises a preservative.

3. The exosome treatment solution according to claim 6-2, wherein the preservative comprises at least one of sodium azide, Proclin 300, 5-bromo-5-nitro-1,3-dioxane, and Proclin 900;
preferably, content of the buffer material in the exosome treatment solution is 2-4% by weight;
preferably, the buffer material comprises trimethylolaminomethane and a phosphate buffer salt;
preferably, content of the trimethylolaminomethane in the exosome treatment solution is 0.9-2.1% by weight;
preferably, content of the phosphate buffer salt in the exosome treatment solution is 1.1-1.9% by weight;
preferably, the phosphate buffer salt comprises at least one of sodium phosphate, potassium phosphate, dipotassium hydrogen phosphate, potassium dihydrogen phosphate, disodium hydrogen phosphate, and sodium dihydrogen phosphate, further preferably a mixture of disodium hydrogen phosphate and sodium dihydrogen phosphate;
preferably, a weight ratio of disodium hydrogen phosphate to sodium dihydrogen phosphate is 1:0.7-1.1;
preferably, the disodium hydrogen phosphate comprises disodium hydrogen phosphate dodecahydrate;
preferably, the sodium dihydrogen phosphate comprises sodium dihydrogen phosphate dihydrate;
preferably, content of the ionic strength maintaining agent in the exosome treatment solution is 0.6-1.2% by weight;
preferably, the ionic strength maintaining agent comprises at least one of potassium chloride, sodium chloride, calcium chloride, and magnesium chloride, further preferably sodium chloride;
preferably, content of the blocking agent in the exosome treatment solution is 0.8-2.2% by weight;
preferably, the blocking agent comprises at least one of casein and collagen peptide, and BSA, further preferably BSA;
preferably, content of the surfactant in the exosome treatment solution is 0.05-0.6% by weight;
preferably, the surfactant comprises a nonionic surfactant; further preferably at least one of a nonionic surfactant containing a polyoxyethylene alcohol structure, a fatty acid ester of polyoxyethylene sorbitol anhydride, polyoxyethylene octyl phenyl ether, and N-D-glucose-N-methylalkylamide; still further preferably at least one of an alcohol ethoxylate, a polyoxyethylene-polyoxyalkylene block copolymer, Tween-20, Tween-40, Tween-80, Triton X-100, Triton X-114, Triton X-305, Triton X-405, Triton X-705, MEGA 8, and MEGA 10; yet still further preferably Tween-20;
preferably, content of the chelating agent in the exosome treatment solution is 0.03-0.25% by weight;
preferably, the chelating agent comprises a water-soluble salt of ethylenediamine tetraacetic acid, further preferably disodium ethylenediamine tetraacetate;
preferably, content of the sugar in the exosome treatment solution is 0.8-2.2% by weight;
preferably, the sugar comprises trehalose;
preferably, content of the preservative in the exosome treatment solution is 0.08-0.22% by weight; and
preferably, the preservative comprises sodium azide or Proclin 300.

4. The exosome treatment solution according to claim 3, wherein the content of the buffer material in the exosome treatment solution is 2.2-3.8% by weight;
preferably, the content of the trimethylolaminomethane in the exosome treatment solution is 1-2% by weight;
preferably, the content of the phosphate buffer salt in the exosome treatment solution is 1.2-1.8% by weight;
preferably, the weight ratio of disodium hydrogen phosphate to sodium dihydrogen phosphate is 1:0.8-1;
preferably, the content of the ionic strength maintaining agent in the exosome treatment solution is 0.8-1% by weight;
preferably, the content of the blocking agent in the exosome treatment solution is 1-2% by weight;
preferably, the content of the surfactant in the exosome treatment solution is 0.1-0.5% by weight;
preferably, the content of the chelating agent in the exosome treatment solution is 0.05-0.2% by weight;
preferably, the content of the sugar in the exosome treatment solution is 1-2% by weight;
preferably, the content of the preservative in the exosome treatment solution is 0.1-0.2% by weight;
preferably, the exosome treatment solution further comprises water, and the amount of water is the balance; and
preferably, a pH of the exosome treatment solution is 6-8, further preferably 7-8.

5. A reagent comprising the exosome treatment solution according to claim 1.

6. A detection kit, comprising the exosome treatment solution according to claim 1, capture antibody-coated magnetic beads, and a luminophore-labeled detection antibody.
wherein the capture antibody and/or the detection antibody specifically bind to the protein.

7. The detection kit according to claim 6, wherein the luminophore comprises at least one of acridinium ester, horseradish peroxidase, and alkaline phosphatase;
preferably, the detection kit further comprises at least one of a luminescent substrate and a protein calibrant.

8. (canceled)

9. A method for detecting the presence or content of an exosomal protein, comprising a step of pretreating exosomes with the exosome treatment solution according to claim 1.

10. The method according to claim 9, wherein the step of pretreating the exosomes with the exosome treatment solution specifically comprises: mixing the exosome treatment solution with the exosomes and reacting, wherein
preferably, the volume ratio of the exosome treatment solution to the exosomes is 1:2-30;
preferably, reaction time is 5-120 min;
preferably, reaction temperature is 25-42° C.;
preferably, the exosome is derived from one of whole blood, serum, plasma, bronchoalveolar lavage fluid, and urine or a combination thereof.

11.-18. (canceled)

19. A method for detecting the presence of an exosomal material, comprising a step of pretreating exosomes with the exosome treatment solution according to claim 4,
wherein the material coprisins at least one of a nucleic acid and a protein.

20. A method for detecting the presence of an exosomal material, comprising a step of pretreating exosomes with the exosome treatment solution according to claim 4,
wherein the material coprisins at least one of a nucleic acid and a protein.

21. A method for diagnosing or assisting in diagnosing a lung disease, comprising a step of detecting the content, expression and/or activity of exosomal CA125 in a test sample,
wherein the lung disease includes pneumonia or an interstitial lung disease; and
wherein the test sample is pretreated with the exosome treatment solution according to claim 4.

22. A method for diagnosing or assisting in diagnosing a lung disease, comprising a step of detecting the content, expression and/or activity of exosomal CA125 in a test sample,
wherein the lung disease includes pneumonia or an interstitial lung disease; and
wherein the test sample is pretreated with the exosome treatment solution according to claim 4.

23. A method for diagnosing or assisting in diagnosing pulmonary fibrosis, comprising a step of detecting the content, expression and/or activity of exosomal CA125 in a test sample,
wherein the test sample is pretreated with exosome treatment solution according to claim 4.

24. The exosome treatment solution according to claim 1, wherein the exosome treatment solution further comprises a preservative.

25. The detection kit according to claim 6, wherein the protein is CA125.

26. The method according to claim 9, wherein the method further comprises the following steps:
(1) mixing the pretreated exosomes with capture antibody-coated magnetic beads, reacting to obtain a capture antibody-coated magnetic bead-exosome complex;
(2) mixing the capture antibody-coated magnetic bead-exosome complex with a luminophore-labeled detection antibody, reacting to obtain a capture antibody-coated magnetic bead-exosome-luminophore-labeled detection antibody complex; and
(3) mixing the capture antibody-coated magnetic bead-exosome-luminophore-labeled detection antibody complex with a luminescent substrate, reacting and detecting the luminous intensity to obtain the protein content in an exosome sample,
preferably, in steps (1) and (2), reaction time is 5-120 min; and
preferably, in steps (1) and (2), reaction temperature is 25-42° C.

27. The method according to claim 26, in step (3), a method for obtaining the protein content in the exosome sample comprises using a protein calibrant to establish a standard curve by a standard curve method, and then calculating the protein content in the exosome sample according to the detected luminous intensity.
